(19) United States
(12) Patent Application Publication
(10) Pub. No.: US 2009/0004720 A1
(43) Pub. Date: Jan. 1, 2009

(54) Smart Biocatalysts For Organic Synthesis

(76) Inventors: Ling Hua, Dallas, TX (US); Dunming Zhu, Dallas, TX (US)

Correspondence Address: BAKER BOTTS L.L.P., 2001 ROSS AVENUE, SUITE 600, DALLAS, TX, 75201-2980, US

(21) Appl. No.: 11/768,352

(22) Filed: Jun. 26, 2007

(51) Int. Cl. C12N 9/14; C07H 21/04

(52) U.S. Cl. 435/195; 536/22.1

(57) ABSTRACT

The present disclosure relates to compositions, systems, and methods that include a reusable biocatalyst. A reusable biocatalytic composition may include a stimulus-responsive support operable to be soluble under at least one first condition and insoluble under at least one second condition; and a biocatalyst bound to the stimulus-responsive support complex. According to some embodiments, a method for catalyzing a reaction may include contacting a first reactant with a composition comprising a stimulus-responsive support, a biocatalyst linked to the stimulus-responsive support, and a first solvent under conditions in which the stimulus-responsive support is soluble in the solvent and the reactant is converted to a product. A reusable biocatalyst system, in some embodiments, may include (a) a stimulus-responsive support; (b) a biocatalyst (i) bound to the stimulus-responsive support complex and (ii) having catalytic activity in the presence of a substrate; and a solvent operable to support both first and second condition.

### TECHNICAL FIELD

The present invention is related to methods, compositions, and systems that include a reusable biocatalyst.

### BACKGROUND OF THE INVENTION

In view of modern environmental concerns, environmentally benign methods for chemical production have become desirable.

Enzyme catalysts are recognized as useful tools for accomplishing industrially important chemical reactions because of their environmentally benign reaction conditions and/or unparalleled stereo-, regio-, and/or chemo-selectivity. Biocatalytic routes offer great promise of radically altering chemical processes for the production of fine chemicals such as pharmaceuticals, agrochemicals, fragrances, flavors, food additives, and consumer care products.

One challenge to fulfilling this promise in biocatalytic processes is the activity and stability of a biocatalyst (e.g., an enzyme). Biomolecular engineering techniques may be employed to enhance one or more properties of an enzyme including, for example, pH stability, thermal stability, and/or catalytic activity. Even where one or more properties of an enzyme may be improved by these methods, it may still be desirable to recover and recycle the improved enzyme (e.g., to control/reduce production costs).

### SUMMARY

Therefore, a need has arisen for improved compositions, systems, and methods for recycling biocatalysts. The present disclosure accordingly relates, in some embodiments, to compositions, systems, and methods for recycling biocatalysts. The present disclosure also relates to compositions, systems, and methods that include a reusable biocatalyst.

For example, a reusable biocatalytic composition may include a stimulus-responsive support operable to be soluble under at least one first condition and insoluble under at least one second condition; and a biocatalyst (a) bound to the stimulus-responsive support complex and (b) having catalytic activity in the presence of a substrate. A stimulus-responsive support may include, for example, (a) a poly(N-alkylacrylamide), wherein the alkyl of N-alkylacrylamide has less than about 20 carbon atoms; (b) a copolymer of N-alkylacrylamide and alkyl acrynoate; and/or (c) a polypeptide. A biocatalyst may include an enzyme (e.g., alcohol dehydrogenase, carbonyl reductase, aldolase, enoate reductase, monooxygenase, dioxygenase, oxynitrilase, nitrilase, lipase, and/or halopexoidase) in some embodiments. A catalytic composition, according to some embodiments, may include a coenzyme (e.g., flavin mononucleotide (FMN), flavinadenine dinucleotide (FAD), nicotinamide adenine dinucleotide (NAD), and/or nicotinamide adenine dinucleotide phosphate (NADP)). In some embodiments of the disclosure, a biocatalyst may include a cofactor, a ligand, and/or a nucleic acid. A biocatalyst, in some embodiments, may be linked to a stimulus-responsive support by a hydrogen bond, an ionic bond, a Van der Waals force, a hydrophobic interaction, and/or a covalent bond. A stimulus-responsive support may have, for example, a critical solution temperature from about 15° C. to about 70° C. and/or a critical solution pH from about 4 to about 10.

According to some embodiments, a method for catalyzing a reaction may include contacting a first reactant with a composition comprising a stimulus-responsive support, a biocatalyst linked to the stimulus-responsive support, and a first solvent under conditions in which the stimulus-responsive support is soluble in the solvent and the first reactant is converted to a first product. A composition may include, for example, a coenzyme, a cofactor, a ligand, and/or a nucleic acid. A method may also include applying a first stimulus (e.g., a temperature change, a pH change, and/or an ionic strength change) to the stimulus-responsive support to precipitate the stimulus-responsive support, in some embodiments. A method, in some embodiments, may further include separating the precipitated stimulus-responsive support from at least the first solvent. According to some embodiments, a biocatalyst may be reused by contacting the precipitated stimulus-responsive support with a second solvent; and applying a second stimulus to the precipitated stimulus-responsive support to dissolve it in the second solvent to form a redissolved biocatalyst—stimulus-responsive support composition. A method may further include contacting a second reagent with the redissolved biocatalyst—stimulus-responsive support composition under conditions that permit formation of a second product in some embodiments. A method may include, according to some embodiments, monitoring the progress of the reactant conversion to product. In some embodiments, a first product and/or a second product may include a pharmaceutical compound and/or a pharmaceutical compound precursor.

A reusable biocatalyst system, in some embodiments, may include (a) a stimulus-responsive support operable to be soluble under at least one first condition and insoluble under at least one second condition; (b) a biocatalyst (i) bound to the stimulus-responsive support complex and (ii) having catalytic activity in the presence of a substrate; and a solvent operable to support both first and second condition.

### DETAILED DESCRIPTION

The present disclosure relates to reusable biocatalysts and compositions, systems, and methods for making and using the same.

### Stimulus-Responsive Support

According to some embodiments of the disclosure a biocatalyst (e.g., an enzyme) may be immobilized on a support. An immobilized catalyst may (a) ease recovery and/or reuse, (b) simplify downstream processing, (c) ease adaptation for continuous operation, and/or (d) improve one or more properties (e.g., catalytic properties) of the biocatalyst.

A biocatalyst (e.g., an enzyme), according to some embodiments, may be immobilized on an insoluble support (e.g., surface of a plate or well, a bead). Reactions carried out by an immobilized biocatalyst on an insoluble support may occur heterogeneously. In some circumstances though, interactions between the enzyme and the matrix may decrease the activity of the enzyme. In addition, the enzyme may not be as physically accessible to reactants when bound to a surface, which may undesirably limit reaction rate and productivity of the bound catalyst. Thus, according to some embodiments, a method, composition, and/or system of the disclosure may exclude a biocatalyst immobilized on or linked to an insoluble support.

Some embodiments of the disclosure include a biocatalyst (e.g., an enzyme) bound to a stimulus-responsive support that is reversibly soluble in a solvent (e.g., water). A stimulus-responsive support may reversibly convert between soluble and insoluble forms as a function of, for example, pH, temperature, ionic strength, and/or the presence/absence of a chemical species. In some embodiments, a biocatalyst on a stimulus-responsive support may carry out a reaction homogeneously or substantially homogeneously (“homogeneously”) (e.g., when a support is dissolved in a solvent). A biocatalyst immobilized on or linked to a stimulus-responsive support may, according to some embodiments, have properties similar to a corresponding non-immobilized enzyme under otherwise similar conditions. For example, a biocatalyst immobilized on a stimulus-responsive support may exhibit similar reversible solubility behaviors in water and other solvents.

In some embodiments, a stimulus-responsive support may convert from a soluble form to an insoluble form and/or from an insoluble form to a soluble form at a temperature (“critical solution temperature”)(CST) of from about 15° C. to about 50° C., from about 15° C. to about 25° C., from about 15° C. to about 30° C., from about 15° C. to about 40° C., from about 15° C. to about 50° C., from about 20° C. to about 40° C., from about 20° C. to about 50° C., from about 25° C. to about 40° C., and/or from about 25° C. to about 50° C.

In some embodiments, the CST may be the same for converting from the insoluble to the soluble form as the CST for converting from the soluble to the insoluble form. In some embodiments, the CST for converting from the insoluble to the soluble form may be different from the CST for converting from the soluble to the insoluble form.

A stimulus-responsive support may convert from a soluble form to an insoluble form and/or from an insoluble form to a soluble form at a pH (“critical solution pH”)(CSpH) of from about 2 to about 12, from about 2 to about 8, from about 6 to about 12, from about 3 to about 11, from about 4 to about 10, and/or from about 5 to about 9, according to some embodiments of the disclosure.

In some embodiments, the CSpH may be the same for converting from the insoluble to the soluble form as the CSpH for converting from the soluble to the insoluble form. In some embodiments, the CSpH for converting from the insoluble to the soluble form may be different from the CSpH for converting from the soluble to the insoluble form.

Ionic strength is a measure of the total concentration of ions in solution and may be calculated as follows:

IS=½Σₙcₙzₙ² (Eq. 1)

wherein c is the concentration of a given ion, z is the valence of a given ion, and n is the number of ion species in the solution. A stimulus-responsive support may convert from a soluble form to an insoluble form and/or from an insoluble form to a soluble form at an ionic strength (“critical solution ionic strength”)(CSIS) of from about 10 μM to about 8 M, from about 10 μM to about 1 M, from about 10 μM to about 1 mM, from about 1 mM to about 1 M, from about 1 mM to about 3 M, and/or from about 10 mM to about 3 M.

In some embodiments, the CSIS may be the same for converting from the insoluble to the soluble form as the CSIS for converting from the soluble to the insoluble form. In some embodiments, the CSIS for converting from the insoluble to the soluble form may be different from the CSIS for converting from the soluble to the insoluble form.

A solvent (e.g., a solvent comprising a stimulus-responsive support) may include, for example, water, an alcohol, a hydrocarbon, a halogenated hydrocarbon, an acid, an ether, an amine, dimethylsulfoxide (DMSO), derivatives thereof, and combinations thereof.

A stimulus-responsive support, in some embodiments, may include any natural or synthetic polymer that undergoes a conformational or phase change in response to a variation in temperature, pH, and/or ionic strength. According to some embodiments, a stimulus-responsive support may include any natural or synthetic polymer that undergoes a conformational or phase change in response to illumination with one or more wavelengths and/or in response to contact with a chemical species. Such polymers may be used in “smart” applications in biology and medicine, ranging from drug delivery systems and cell adhesion mediators to the controllers of enzyme function and gene expression.

A stimulus-responsive support may include, without limitation, a poly(N-alkylacrylamide), a copolymer of an N-alkylacrylamide) and alkyl acrynoate, and/or a polypeptide. An alkyl may include a branched or unbranched alkyl group with less than about 20 carbon atoms.

Poly(N-isopropylacrylamide) (PNIPAAm) is a thermo-reversible water-soluble polymer, which exhibits a lower critical solution temperature (LCST) of 32° C. The polymer precipitates from aqueous solution when it is heated above the LCST, and is re-dissolved when cooled below that temperature. This process is fully reversible. The LCST may be tuned as desired by variation in the N-isopropylacrylamide/co-monomer contents.

According to some embodiments of the disclosure, these PNIPAAm-based and other stimuli-responsive polymers may be used to conjugate with biocatalysts (e.g., synthetically important enzymes) with the aim of developing smart biocatalysts for the synthesis of fine chemicals.

### Biocatalysts

According to some embodiments, a biocatalyst may include an enzyme. For example, a biocatalyst may include an acetyltransferase, an acyltransferase, an aldolase, an ATPase, a carbamoyltransferase, a carboxylase, a carboxylic ester hydrolase, a carboxytransferase, a collagenase, a convertase, a cyclase, a deaminase, a decarboxylase, a dehalogenase, a dehydrogenase, a desaturase, a dioxygenase, a dismutase, an elastase, an epimerase, a glycosidase, a GTPase, a hydratase, a hydrogenase, a hydrolase, a hydroxylase, an isomerase, a ketoreductase, a kinase, a ligase, a lipase, a lipoxygenase, a lyase, a methylase, a methyltransferase, a mutase, a nitrilase, a nitrogenase, a nuclease, a nucleosidase, an oxidase, an oxygenase, a peptidase, a phosphatase, a phosphodiesterase, a phosphorylase, a polymerase, a protease, a proteinase, a racemase, a reductase, a sulfatase, a synthase, a synthetase, a tautomerase, a thiolase, a transaldolase, a transaminase, a transketolase, a variant thereof, and/or a derivative thereof.

A biocatalyst may include a catalytic antibody, a catalytic nucleic acid, and/or combinations thereof in some embodiments.

A biocatalyst, in some embodiments, may include a coenzyme, a cofactor, a ligand, and/or a nucleic acid. An enzyme, a coenzyme, a cofactor, a ligand, a nucleic acid, and/or a catalytic nucleic acid may alternatively or additionally be present in a solvent in which a biocatalyst is dissolved.

A coenzyme, in some embodiments, may include adenosine diphosphate (ADP), adenosine monophosphate (AMP), adenosine triphosphate (ATP), ascorbic acid (Vitamin C), biotin, coenzyme A (CoA), coenzyme B₁₂, coenzyme Q, flavin mononucleotide (FMN), flavinadenine dinucleotide (FAD), folic acid, iron protoporphyrin (hemin), molybdopterin, nicotinamide adenine dinucleotide (NAD), nicotinamide adenine dinucleotide phosphate (NADP), riboflavin (B₂), thiamine pyrophosphate (TPP), UDP-glucose, uridine diphosphate (UDP), variants thereof, and/or derivatives thereof.

A cofactor may include manganese, iron, cobalt, nickel, copper, zinc, molybdenum, vanadium, and/or tungsten according to some embodiments of the disclosure. A cofactor, in some embodiments, may include a porphyrin ring.

In some embodiments, a nucleic acid may include any natural or artificial mono, oligo, poly nucleotide, variants thereof, and/or derivatives thereof. A nucleic acid may include a nucleoprotein complex.

A biocatalyst may be bound to a support (e.g., a stimulus-responsive support) by one or more of the following: a hydrogen bond, an ionic bond, a Van der Waals force, a hydrophobic interaction, and/or a covalent bond. A biocatalyst, in some embodiments, may be bound to a support (e.g., a stimulus-responsive support) via a linker. A linker may include, for example, an alkyl chain, an ether, an amine, an ester, and/or an aromatic group.

### Methods

According to some embodiments, a method of the disclosure may include contacting a reactant with a composition comprising a stimulus-responsive support, a biocatalyst linked to or immobilized on the stimulus-responsive support, and a solvent under conditions in which the stimulus-responsive support is soluble in the solvent. A method may further include applying a stimulus to the composition to convert the stimulus-responsive support to its corresponding insoluble form, in some embodiments. A method of the disclosure may, in some embodiments, further include separating the biocatalyst—stimulus-responsive support from the reagent and/or the solvent. In some embodiments, a method may further include subsequently contacting the separated biocatalyst—stimulus-responsive support with a second reagent (e.g., the same as or different from the first reagent) and/or a second solvent (e.g., the same as or different from the first solvent).

For example, a method may include running a reaction to completion. Then, when the reaction is complete, an appropriate stimulus such as change of pH, temperature, ionic strength or addition of a chemical species is applied to the reaction system, and the biocatalyst—-stimulus-responsive support forms a precipitate. This precipitate may be separated from soluble and/or suspended reaction products, e.g., products may be isolated from the supernatant. The recovered enzymes can be re-dissolved in a buffer for further reaction with fresh substrate.

In some embodiments, a method may include a formation of an insoluble reaction product, for example, a product that precipitates. An insoluble reaction product may be separated from the soluble form of a biocatalyst—stimulus-responsive support complex. Once that product is removed, a stimulus may be applied to remaining solvent to precipitate the biocatalyst—stimulus-responsive support complex.

Thus, in some embodiments, a biocatalyst may be recovered and reused. Some embodiments of the disclosure may take advantage of both homogeneous and heterogeneous catalytic processes, and may also ameliorate one or more of the drawbacks of heterogeneous reactions.

A method, in some embodiments, includes linking or immobilizing a biocatalyst (e.g. an enzyme, a catalytic antibody, a catalytic RNA) on a stimulus-responsive support, contacting the biocatalyst—stimulus-responsive support complex with a solvent under conditions that permit the complex to dissolve.

In some embodiments, a method may include formation of a pharmaceutical compound and/or a pharmaceutical compound precursor. A pharmaceutical compound and/or a pharmaceutical compound precursor may include any compound with pharmaceutical activity in at least one mammal (human or non-human). A pharmaceutical compound and/or a pharmaceutical compound precursor may include 1-cyanocyclohexaneacetic acid, which may be a precursor for a drug with anti-depressant properties (e.g., gabapentin (a.k.a., Neurontin)). A pharmaceutical compound may include a compound with anti-convulsant properties (e.g., pregabalin (a.k.a. Lyrica) and/or cholesterol lowering properties (e.g., atorvastatin (a.k.a. Lipitor)). A pharmaceutical compound may include, in some embodiments, a compound with anti-obesity properties (e.g., orlistat (a.k.a. Xenical)). In some embodiments, a pharmaceutical may include a β₂-agonist. A β₂-agonist may be used in the management of asthma and/or chronic obstructive pulmonary disease (COPD) (e.g., (R,R)-formoterol (a.k.a. Foradil/Foradile)).

### Systems

A system of the disclosure, according to some embodiments, may include a biocatalyst, a stimulus-responsive support, and a solvent. A system may also include one or more of the following:

a vessel configured and arranged to contain at least a portion of a biocatalyst, a stimulus-responsive support, and a solvent;

one or more solvents, each independently the same or different from the others, for concurrent (e.g., mixed or unmixed) or sequential use;

one or more biocatalysts, each independently attached to common or separate supports (e.g., stimulus-responsive supports);

one or more cofactors, coenzymes, ligands, and/or nucleic acids, each independently attached to common supports, separate supports or suspended (e.g., dissolved) in a solvent; and/or

instruments to separate and reuse a biocatalyst (e.g., pipet(s), centrifuge(s), and/or microfluidic devices).

### EXAMPLES

### Example 1

A copolymer containing N-isopropylacrylamide (NIPAAm) and N-acryloxysuccinimide (NAS) was prepared by the copolymerization of NIPAAm and NAS (the molar ratio of NIPAAm to NAS is 40) in the THF/toluene mixture. The polymerization was initiated by the addition of 2,2′-azobis(isobutyronitrile) (AIBN). The polymer was isolated by precipitation with hexane and dried under vacuum. The obtained polymer had an LCST of about 33° C. This polymer had active ester groups, which were highly reactive to primary amino groups in protein molecules.

### Example 2

Immobilization of a Carbonyl Reductase. A carbonyl reductase from Sporobolomyces salmonicolor (SSCR) was treated with the poly(N-isopropylacrylamide) (NIPAAm) in potassium phosphate buffer (100 mM, pH 8.0) at room temperature for 2 hours. After addition of NaCl, a white precipitate was isolated and washed with buffer containing same concentration of NaCl. The precipitation can also be induced by increasing the temperature above 33° C. (LCST). The protein concentrations and activity of both the isolated immobilized enzyme and the supernatant were measured. The coupling yield of protein was defined as the amount of protein immobilized to that added initially during the immobilization step. The coupling yield of enzyme activity was defined as the enzyme activity of the immobilized protein to that of the initially added protein. The relative specific activity was defined as the specific activity of the immobilized enzyme to that of the free enzyme. It was found that the coupling yield of protein was 53% and the relative specific activity of the immobilized enzyme was 72%. The coupling yield of enzyme activity was about 38%.

### Example 3

Immobilization of D-Glucose Dehydrogenases. A D-glucose dehydrogenase (GDH, which reduces NADP⁺ to NADPH for regeneration of cofactor NADPH) was treated with poly(NIPAAm) in potassium phosphate buffer (100 mM, pH 8.0) at room temperature for 2 hours. After addition of NaCl, a white precipitate was isolated and washed with buffer containing same concentration of NaCl. The precipitation can also be induced by increasing the temperature above 33° C. (LCST). The protein concentrations and activity of both the isolated immobilized enzyme and the supernatant were measured. The coupling yield of protein and the relative specific activity of the immobilized enzyme were found to be 47% and 75%, respectively. The coupling yield of enzyme activity was about 35%.

### Example 4

Co-Immobilization of a Carbonyl Reductase and a D-Glucose Dehydrogenase. A mixture of a carbonyl reductase from Sporobolomyces salmonicolor (SSCR) and a D-glucose dehydrogenase (GDH) was treated with poly(NIPAAm) in potassium phosphate buffer (100 mM, pH 8.0) at room temperature for 2 hours. After addition of NaCl, a white precipitate was isolated and washed with buffer containing same concentration of NaCl. The precipitation can also be induced by increasing the temperature above 33° C. (LCST).

### Example 5

Reduction of Ketones. The enzyme activity and enantioselectivity of immobilized SSCR were tested with ethyl 3,3-dimethyl-2-oxobutyrate as the substrate. Ethyl 3,3-dimethyl-2-oxobutyrate was treated with immobilized SSCR in potassium phosphate. The co-factor NADPH was regenerated with a D-glucose/D-glucose dehydrogenase system. The immobilized SSCR was repeatedly used for six times. Each time ethyl 3,3-dimethyl-2-oxobutyrate was reduced to ethyl(R)-3,3-dimethyl-2-hydroxybutyrate in optically pure form at 100% conversion. After being used for six times, the immobilized SSCR was assayed by spectrophotometrically measuring the oxidation of NADPH at 340 nm (ε=6.22 mM⁻¹ cm⁻¹), and it was found that 70% of activity was maintained. These results indicate that the immobilized SSCR enzyme possesses excellent enantioselectivity and can be repeatedly used.

The reduction of ethyl 3,3-dimethyl-2-oxobutyrate was also performed with immobilized SSCR and immobilized GDH under the same conditions. The immobilized SSCR and immobilized GDH were used for six cycles, and each time ethyl 3,3-dimethyl-2-oxobutyrate was reduced to ethyl (R)-3,3-dimethyl-2-hydroxybutyrate in optically pure form at 100% conversion. The activity assay was difficult to be performed by spectrophotometrically measuring the oxidation of NADPH at 340 nm due to the interference of SSCR and GDH.

The reduction of ethyl 3,3-dimethyl-2-oxobutyrate was then performed with co-immobilized SSCR and GDH under the same conditions. The co-immobilized SSCR and GDH were used for six cycles, and each time ethyl 3,3-dimethyl-2-oxobutyrate was reduced to ethyl(R)-3,3-dimethyl-2-hydroxybutyrate in optically pure form at 100% conversion. The activity assay was also difficult to be performed by spectrophotometrically measuring the oxidation of NADPH at 340 nm due to the interference of SSCR and GDH.

In summary, these polymer-supported biocatalysts were soluble in water at room temperature, but precipitated at the temperature above 32° C. or with addition of salts such as sodium chloride. It has been found that the activity and stereoselectivity towards the reduction of ethyl 3,3-dimethyl-2-oxobutyrate were comparable to the free enzymes. Furthermore, these immobilized biocatalysts did not show significant loss in activity and stereoselectivity after six cycles (i.e., after reuse six times).

### Example 6

Immobilization of a nitrilase. A nitrilase bll6402 from Bradyrhizobium japonicum USDA110 was treated with the polymer in potassium phosphate buffer (100 mM, pH 8.0) at room temperature for 2 hours. After addition of NaCl, a white precipitate was isolated and washed with buffer containing same concentration of NaCl. The precipitation can also be induced by increasing the temperature above 33° C. (LCST). The protein concentrations and activity of both the isolated immobilized enzyme and the supernatant were measured. The coupling yield of protein was defined as the amount of protein immobilized to that added initially during the immobilization step. The coupling yield of enzyme activity was defined as the enzyme activity of the immobilized protein to that of the initially added protein. The relative specific activity was defined as the specific activity of the immobilized enzyme to that of the free enzyme. It was found that the coupling yield of protein was 63% and the relative specific activity of the immobilized enzyme was 74%. The coupling yield of enzyme activity was about 47%.

As will be understood by those skilled in the art with the benefit of the instant disclosure, other equivalent or alternative composition, systems, and methods related to reusable biocatalysts can be envisioned without departing from the essential characteristics of the present disclosure. For example, composition, systems, and methods, in some embodiments, may be practiced on a nano, micro, or macro scale. Methods and systems of the disclosure may be practices, for example, in either a handheld or a tabletop configuration, and may be operated sporadically, intermittently, and/or continuously. According to some embodiments, one or more detectors and/or indicators may be used to monitor and/or assess reaction progress and/or completion. One or more detectors and/or indicators may be used to monitor and/or assess the solution state and/or conformation of a stimulus-responsive support.

At least a portion of a system or device of the disclosure may be configured and arranged to be disposable, reusable, repairable, restorable, and/or sterilizible. Moreover, one of ordinary skill in the art with the benefit of the instant disclosure will appreciate that no embodiment, use, and/or advantage described in this disclosure is intended to universally control or exclude other embodiments, uses, and/or advantages. These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

What is claimed is:

1. A reusable biocatalytic composition, said composition comprising:
a stimulus-responsive support operable to be soluble under at least one first condition and insoluble under at least one second condition; and
a biocatalyst (a) bound to the stimulus-responsive support complex and (b) having catalytic activity in the presence of a substrate.

2. A catalytic composition according to claim 1, wherein the stimulus-responsive support is selected from the group consisting of (a) poly(N-alkylacrylamide), wherein the alkyl of N-alkylacrylamide has less than about 20 carbon atoms; (b) a copolymer of N-alkylacrylamide and alkyl acrynoate; and (c) a polypeptide.

3. A catalytic composition according to claim 1, wherein the biocatalyst comprises an enzyme.

4. A catalytic composition according to claim 3, wherein the enzyme is selected from the group consisting of alcohol dehydrogenase, carbonyl reductase, aldolase, enoate reductase, monooxygenase, dioxygenase, oxynitrilase, nitrilase, lipase, and halopexoidase.

5. A catalytic composition according to claim 1, wherein the biocatalyst comprises an coenzyme.

6. A catalytic composition according to claim 5, wherein the coenzyme is selected from the group consisting of flavin mononucleotide (FMN), flavinadenine dinucleotide (FAD), nicotinamide adenine dinucleotide (NAD), and nicotinamide adenine dinucleotide phosphate (NADP).

7. A catalytic composition according to claim 1, wherein the biocatalyst comprises a molecule selected from the group consisting of a cofactor, a ligand, and a nucleic acid.

8. A catalytic composition according to claim 1, wherein the biocatalyst is linked to the stimulus-responsive support by a bond selected from the group consisting of a hydrogen bond, an ionic bond, a Van der Waals force, a hydrophobic interaction, and a covalent bond.

9. A catalytic composition according to claim 1, wherein the stimulus-responsive support has a critical solution temperature is from about 15° C. to about 70° C.

10. A catalytic composition according to claim 1, wherein the stimulus-responsive support has a critical solution pH is from about 4 to about 10.

11. A method for catalyzing a reaction, said method comprising:
contacting a first reactant with a composition comprising a stimulus-responsive support, a biocatalyst linked to the stimulus-responsive support, and a first solvent under conditions in which the stimulus-responsive support is soluble in the solvent and the first reactant is converted to a first product.

12. A method according to claim 11 further comprising applying a first stimulus to the stimulus-responsive support to precipitate the stimulus-responsive support.

13. A method according to claim 12, wherein the stimulus is selected from the group consisting of a temperature change, a pH change, and an ionic strength change.

14. A method according to claim 12 further comprising separating the precipitated stimulus-responsive support from at least the first solvent.

15. A method according to claim 14 further comprising:
contacting the precipitated stimulus-responsive support with a second solvent; and
applying a second stimulus to the precipitated stimulus-responsive support to dissolve it in the second solvent to form a redissolved biocatalyst—stimulus-responsive support composition.

16. A method according to claim 15 further comprising:
contacting a second reagent with the redissolved biocatalyst—stimulus-responsive support composition under conditions that permit formation of a second product.

17. A method according to claim 11, wherein the composition further comprises a molecule selected from the group consisting of a coenzyme, a cofactor, a ligand, and a nucleic acid.

18. A method according to claim 11 further comprising monitoring the progress of the reactant conversion to product.

19. A method according to claim 11, wherein the product is a pharmaceutical compound or a pharmaceutical compound precursor.

20. A reusable biocatalyst system, said system comprising:
a stimulus-responsive support operable to be soluble under at least one first condition and insoluble under at least one second condition;
a biocatalyst (a) bound to the stimulus-responsive support complex and (b) having catalytic activity in the presence of a substrate; and
a solvent operable to support both first and second condition.
